(19) United States
(12) Patent Application Publication
(10) Pub. No.: US 2025/0340675 A1
(43) Pub. Date: Nov. 6, 2025

(54) NEW HYALURONIC ACID DERIVATIVES AS INNOVATIVE FILLERS

(71) Applicant: WiQo S.P.A., Milano (MI) (IT)

(72) Inventor: Rossana Castellana, Trieste (IT)

(73) Assignee: WiQo S.P.A., Milano (MI) (IT)

(21) Appl. No.: 18/727,346

(22) PCT Filed: Jan. 10, 2023
(86) PCT No.: PCT/EP2023/050456

§ 371 Date: Jul. 9, 2024

Related U.S. Application Data
(60) Provisional application No. 63/298,224, filed on Jan. 11, 2022.

(51) Int. Cl. C08B 37/08; A61K 31/167; A61K 31/728; C07C 69/732; C07H 15/207

(52) U.S. Cl. CPC C08B 37/0072; A61K 31/167; A61K 31/728; C07C 69/732; C07H 15/207

(57) ABSTRACT

The present invention relates to new hyaluronic acid derivatives conjugated with molecules of natural origin endowed with anti-inflammatory and/or antioxidant properties and processes for manufacturing the same. The covalent link of these bioactive molecules to hyaluronic acids and the degree of reticulation with the same affords peculiar characteristics in term of controlled release of the bioactive molecules and of viscoelastic properties and stabilize the obtained hyaluronic derivatives to the chemical and to the enzymatic degradation. These new hyaluronic acid derivatives are useful for the preparation of injectable dermal filler compositions effective as soft tissue fillers, like dermal and subdermal fillers.

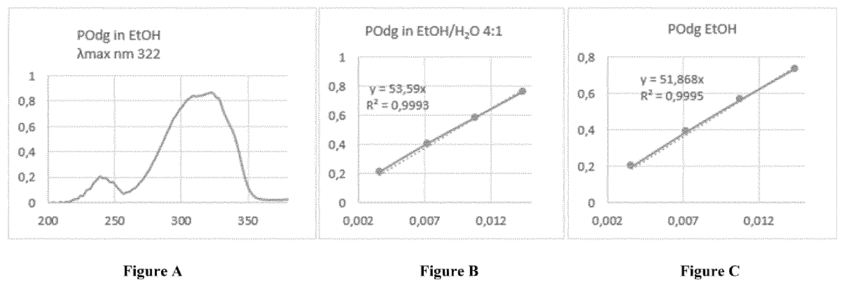

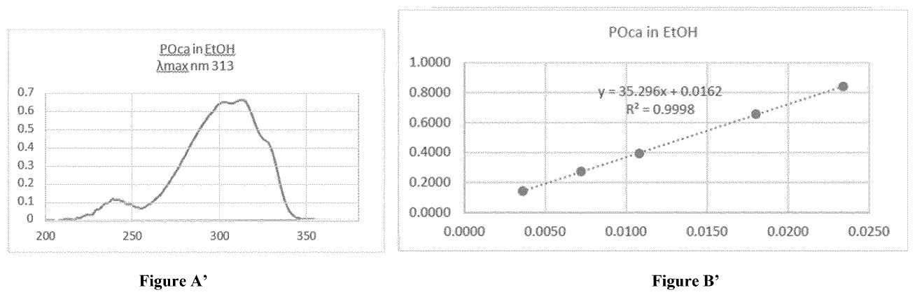

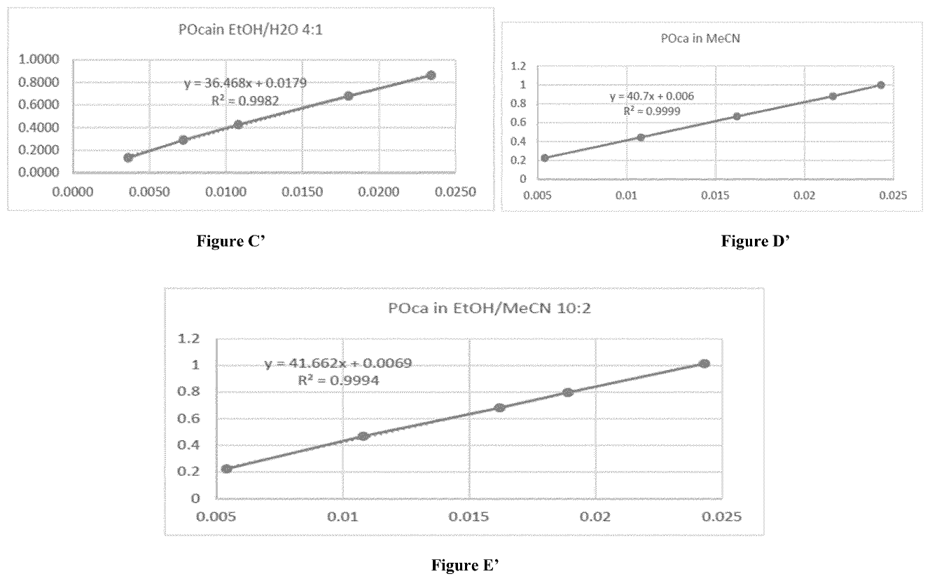

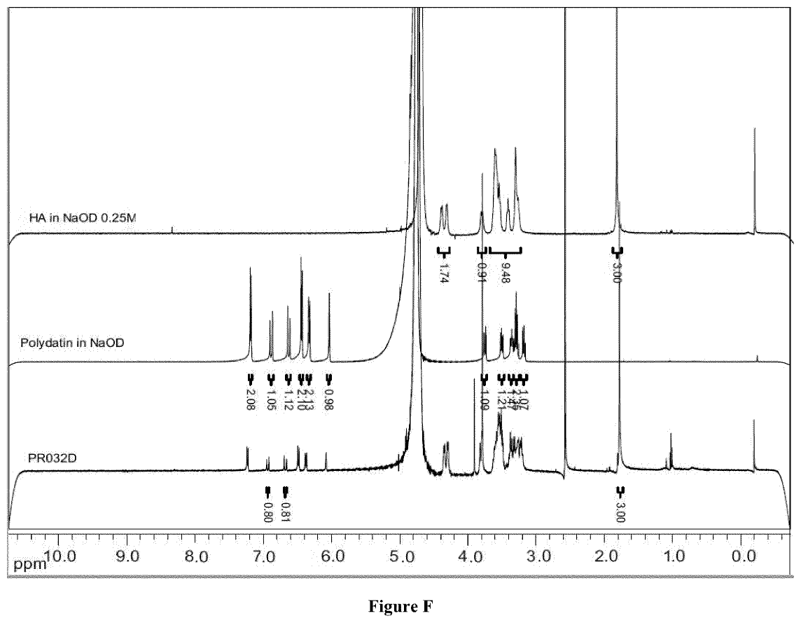

This application is a U.S. national stage of PCT/EP2023/050456 filed on 10 Jan. 2023, which claims priority to and the benefit of U.S. Provisional Application No. 63/298,224 filed on 11 Jan. 2022, the contents of which are incorporated herein by reference in their entireties.

### TECHNICAL FIELD

The present invention relates to the field of polysaccharides. More specifically, the present invention is concerned with novel processes of cross-linking hyaluronic acid (HA) of different molecular weight with functionalized molecules of natural origin endowed with anti-inflammatory and/or antioxidant properties and of manufacturing cross-linked HA products. Injectable monophasic gels containing hyaluronic acid derivatives obtained by these processes may be utilized as tissue fillers and for tissue augmentation in the field of cosmetic surgery and medical aesthetic.

### BACKGROUND OF THE INVENTION

Hyaluronic acid (HA) is a polysaccharide that consists of repeating monomers (glucuronic acid sodium salt and N-acetylglucosamine disaccharide units) linked together in a linear fashion through β-1,4 glycosidic bonds and belong to the class of glycosaminoglycans having the following structure (Formula 1).

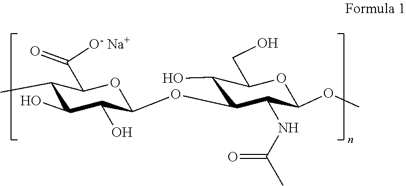

HA is a naturally occurring polymer found in the extracellular matrix, the vitreous humour, and the cartilage. The total quantity of HA found in a normal weight person (70 kg) is approximately 15 g, and its average turnover rate is 5 g/day. Approximately 50% of the total quantity of HA in the human body is present in the skin, and it has a half-life of 24÷48 hours. HA is one of the fundamental components of animal tissues: at the skin level it is present both in free form and combined with proteins. HA gives the skin hydration thanks to its ability to retain water and tonicity thanks to its properties of aggregating the extracellular matrix (the substance “compacting” the dermis). Its deficiency causes a weakening of the “scaffolding” of the skin with a consequent reduction in tone, hydration and resistance. The basis for what can be considered, according to a purely aesthetic canon, the “formation of wrinkles”.

HA as such has no biomechanical properties since in the presence of water is a liquid and not a gel. The injection of a solution of HA as such under a wrinkle to lift it have no effect, moreover, being liquid, it would be absorbed by the tissues within a few hours. Pure HA is used as an injection in the so-called “bio stimulation”: being liquid, it is a stimulus on its own and is rapidly absorbed in the tissue where it is injected.

In order to obtain a gel able of supporting the weight of a tissue and lifting it (in the case of a skin wrinkle, but also in the case of a deteriorated joint such as that of knee) HA must be chemically transformed into a gel. The hyaluronic gel of the fillers (those that are marketed and used as medical devices) are prepared through industrial processes to acquire biomechanical properties (viscosity and elasticity) and to integrate into the tissues.

During the manufacturing process of a hyaluronic gel filler a chemical linker is used, also called cross-linking. One of the most used linker is 1,4-butanediol diglycidyl ether (BBDE) able to create bonds (more or less stable) between the hyaluronic acid filaments (Dermatol Surg 2013; 39:1758-1766; DOI: 10.1111/dsu.12301). The strands bound together become stable, like a compact network, and the whole becomes a solid gel. Cross-linked hyaluronic acids are typical viscous, gelatinous to the touch and are endowed with elastic properties and various degrees of hardness or softness “tailormade” depending by the intended injection site (ie cheek, lips, nasolabial fold, etc). After the injection these fillers showed variable duration, quantified in months they integrate with tissues, giving them “a shape” and “last over time”. The number of cross-linking molecules and the type of bond they form will make the gel soft, dense or hard: stronger and numerically the higher the bonds, the greater the rigidity and hardness of a gel; on the contrary, weak and numerically lower bonds will make the gel softer. The peculiar rheological characteristics of these crosslinked HA fillers can be measured by their elastic modulus (G′), by their viscosity (G″) and swelling factor (SwF) (Barnes H A; Handbook of Elementary Rheology, Institute of Non-Newtonian Fluid Mechanics, University of Wales, 2000); for this last rheological parameter there are no clinical data linking swelling factor and post-treatment swelling, because the factors that can contribute to determine the swelling of the tissues can be various proprietary cross-linking technologies, techniques of injection, quality of tissues, etc.

Examples of hyaluronic acid gel fillers prepared using as cross-linking BBDE are disclosed in the international patent applications WO2017/016917 and WO2005/097218; WO2012/062775, WO2013/028904, WO2013/040242, WO2016/051219 and WO2009/018076; WO2017/001056, WO2017/162676, WO2016/074794, WO2013/185934, WO2017/001057, WO2018/083195 and WO2017/076495.

Although the metabolism of hydrolysed BDDE is not described in the literature, it is understood to proceed through ether bond cleavage by a family of enzymes called cytochromes P450. These enzymes are involved in the oxidative degradation of organic molecules and can catalyse the cleavage of ether bonds into alcohols. After degradation, two main products can emerge: glycerol and 1-4-butanediol. Similar to all diol-ethers, hydrolysed BDDE is also known to be eliminated in urine (Dermatol Surg 2013; 39:1758-1766). 1,4-Butanediol is known to be non-mutagenic, non-sensitizing, and a slight irritant (Ishikawa K. 1,4-butanediol. OECD SIDS CAS N° 110-63-4 2000:1-60; NICNAS 1,4-butanediol. Existing chemical hazard assessment report ISBN 978-O-9803124-7-8 2009. pp. 1-25). No carcinogenic potential has been identified by tests performed on its metabolites. Neurotoxic adverse effects were observed in animals with a no observed adverse effect level (NOAEL) of 100 mg/kg per day (determined according to oral administration in mice). The median lethal dose (LD50) of 1,4-butanediol is 1,525 mg/kg (determined according to oral administration in mice). However, the long-term effects of 1-4 butanediol, which is a synthetic compound utilize as industrial solvent are unknown. What is known is that, when ingested, it is converted to γ-hydroxybutyrate, a drug of abuse with depressant effects, primarily on the central nervous system (N Engl J Med, Vol. 344, No. 2 ⋅ Jan. 11, 2001, 87-94).

Other cross-linking agents utilized for the preparation of hyaluronic gel fillers includes: boronic acid derivatives belonging to the class of alkylboronic hemiesters which produce reversible bonds (WO2018/024795); diamines and polyamines (hexamethylenediamine, lysine monomethyl ester and 3-[3-(3-aminopropoxy)-2,2-bis (3-amino-propoxymethyl)-propoxy]-propylamine) and the carbodiimide (WO2013/040242); citric acid (WO2018/087272); endogenous amines, like spermine and spermidine and as coupling agent N-ethyl,N-(dimethylamminopropyl)-carbodiimide (WO2014/064632); divinyl sulfone (WO2005/066215); hyaluronic acid gels obtained by self-assembly, where the carboxylic groups are activated to react with alcoholic groups present on the same polysaccharide chain or on others nearby (EP0341745); multicomponent condensation products obtained by reaction involving the carboxy groups and the amino groups originating from a partial N-deacetylation of HA or derivatives, together with an aldehyde and an isocyanide (WO0218450); formaldehyde, glutaraldehyde, divinyl sulfone, polyanhydrides, polyaldehydes, polyhydric alcohols, carbodiimides, carboxylic acid chlorides, sulfonic acid chlorides, epichlorohydrin, ethylene glycol, butanediol diglycidyl ether, diglycidyl ether, polyglycerol polyglycidyl ether, polyethylene glycol diglycidyl ether, polypropylene glycol diglycidyl ether and bis- or polyepoxides, preferably in the presence of butanediol diglycidyl ether or divinyl sulfone (EP1837347).

KR 2018 0010361 discloses cross-linked hyaluronic acids obtained by reaction of hyaluronic acids with 1,4-butanediol diglycidyl ether (BDDE) and catechin. The cross-linking bonds are of ether type.

KR 2016 0031081 discloses hyaluronic acids functionalized with polyphenols wherein the polyphenol moiety does not act as a cross-linker.

Another characteristic of HA as dermal filler is its rapid degradation under physiological conditions. The degradation of HA can be explained as a depolymerization process that is mediated by glycosidic bonds cleavage. This depolymerization may precede the dissociation of the polymer chains on a macromolecular level (dissolution and diffusion). The depolymerization of HA has been well characterized in the literature and mainly involves two mechanisms: enzymatic degradation and free radical degradation. A large class of enzymes collectively known as hyaluronidases mediates enzymatic degradation of HA, moreover several reports in the literature indicate that free radical mediated degradation of HA proceeds through cleavage of glycosidic bonds. HA catabolism takes place in situ (e.g., in the extracellular matrix), intracellularly, or after transfer to the lymph nodes and transforms long HA chains (polysaccharides) into smaller HA units (oligosaccharides). Two separate studies using a variety of BDDE-crosslinked HA fillers with different physicochemical properties showed that the BDDE modification does not interfere with the natural enzymatic degradation mechanisms of HA (Jones D, et al. Dermatol Surg 2010; 36:804-9. Sall I et al. Polym Degrad Stab 2007; 92:915-9).

In recent years, the use of dermal fillers has significantly increased: from 650.000 in 2000 to greater than 2.4 million in 2015 per year and this has consequently led to an increase in complications (American Society of Plastic Surgeons, 2014 Plastic surgery statistics report. https://www.plasticsurgery.org/news/plastic-surgery-statistics ?sub=2014+Plastic+Surgery+Statistics. Accessed Jun. 1, 2017). In most cases, fillers are used without clinically significant complications to the patient, although depending on an increase in use and a large variability in clinician training and experience, the overall number of complications has risen (Haneke E. Managing complications of fillers: rare and not-so-rare. J Cutan Aesthet Surg. 2015; 8(4): 198-210). According to the US Food and Drug Administration (FDA), manufacturer and user device experience (MAUDE) database the complications of the HA based fillers, Restylane®, Belotero®, Juvederm®, and Juvederm Voluma® comprise: swelling, infection and nodule formation.

Even if these complications are estimated 0.01% of all injections for HA fillers the necessity of safer HA based fillers is required as well as the possibility to develop new crosslinked HA fillers endowed of enhanced characteristics of safety, stability to depolymerization and tailor made rheologic characteristic. In fact, the long-term effects of some known metabolites of some totally synthetic cross-linking agents, like for example 1-4 butanediol potentially generated by BDDE, are not fully elucidated and promote the research of safer and naturally derived crosslinking agents.

### DESCRIPTION OF THE INVENTION

The present invention discloses new processes for manufacturing a cross-linked hyaluronic acid (HA) gel product, which meets the following requirements: efficient incorporation of cross-linking agent endowed with anti-inflammatory and/or antioxidant properties, sufficient gel strength to resist deformation and migration when implanted and increase stability to thermal treatments of sterilization and enzymatic hydrolysis respect to the employed native HAs. The present invention therefore allows for manufacturing of a gel having enhanced strength with respect to a non-cross-linked HA and a limited swelling degree with a surprisingly low chemical modification of the HA.

It is a further object of the present invention to provide a process with a modular efficiency of the cross-linking reaction. It is a further object of the present invention to minimize the degree of modification that is needed to obtain a HA gel product having a desired gel strength. It is a further object of the present invention to obtain a HA gel product having an enhanced in vivo duration respect to a non-cross-linked HA and at the same time a limited degree of structural modification. It is also an object of the invention to obtain a HA gel product with useful implantation properties, including viscoelastic gel properties and purity from side products and residuals.

The claimed cross-linked hyaluronic acid (HA) gel products according to the present invention were prepared from three different types of hyaluronic acid with the following molecular weights:
low molecular weight fraction: 8-15 kDa-preferentially used for conjugation and formation of a new non-crosslinked derivative or used as part of the crosslinked matrix conferring more viscous properties
fraction at intermediate molecular weight: 500-750 kDa—these fractions may undergo to a targeted purification with cross-flow filtration in order to refine the molecular weight range and bring it as close as possible to the upper limit of the same or used as they are, for crosslinking
high molecular weight fraction: 1.5-3.0 MDa—these fractions were used to complete the formulation with the function of supporting the structure of the filler itself conferring more elastic properties.

For the preparation of this new trimodal (or tricomponent) filler natural and safe bioactive agents like polydatin, gallic acid, chlorogenic acid and phlorizin were utilized as derivatives or crosslinkers.

These compounds have the common features to be natural molecules, commonly present in food and beverages, endowed with anti-inflammatory and/or antioxidant properties, to be water soluble and to have suitable functional groups (ie, hydroxylic and/or carboxylic functional groups) useful for a subsequent modification and consequent bond with hyaluronic acid.

In particular, polydatin (chemical name: β-D-glucopyranoside, 3-hydroxy-5-[2-(4-hydroxyphenyl)ethenyl]phenyl; Formula 2) is the major component of grape juice and the most abundant form of resveratrol in nature. This molecule has shown a wide range of biological activities including anti-inflammatory, anti-oxidant, anti-cancer, neuroprotective, hepatoprotective, nephroprotective and immunostimulatory effects (Didem Sohretoglu et al., Recent advances in chemistry, therapeutic properties and sources of polydatin. Phytochemistry Reviews volume 17, 973-1005 (2018)).

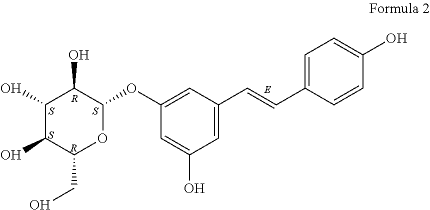

This molecule, a stilbenoid, is a trans-resveratrol substituted in position 3 with a β-D-glucoside residue. Polydatin has 6 hydroxyl groups, two of which are phenolic-type variously reactive which can be used as anchor points for subsequent derivatizations. The presence of the double bond directs the activity since the trans form, unlike the cis form, is biologically active. The derivatizations were addressed to modify two hydroxyl groups (phenolic moieties) or all the hydroxyl groups, in such a way as to be able to use the molecule both as a crosslinker and as a derivatized of the hyaluronic acid chain.

Gallic acid (chemical name: 3,4,5-trihydroxy benzoic acid; Formula 3) is a naturally occurring secondary metabolite found in various plants, vegetables, nuts and fruits like gallnuts, sumac, witch hazel, tea leaves and oak bark.

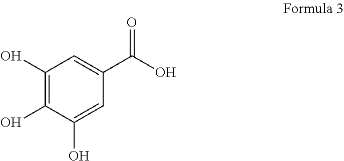 [SMILES: O=C(O)c1cc(O)c(O)c(O)c1]

Gallic acid is a compound endowed with anti-inflammatory and/or anti-oxidative activities and, on the basis of the available literature data, has hardly shown toxicity in animals or clinical trials, thus making it potentially useful for long-term use in inflammation-related diseases (Nouri, F. Heibati, E. Heidarian, Gallic acid exerts anti-inflammatory, anti-oxidative stress, and nephroprotective effects against paraquat-induced renal injury in male rats, Naunyn Schmiedebergs Arch. Pharmacol. 2020). Literature toxicity data confirm that gallic acid is safe for most cells at lower concentrations showing toxic effects only at relatively higher concentrations: the acute toxicity of gallic acid in albino mice showed that the LD50 was greater than 2000 mg/kg (B. C. Variya, et al., Acute and 28-days repeated dose sub-acute toxicity study of gallic acid in albino mice, Regul. Toxicol. Pharmacol 101 (2019) 71-78).

Chlorogenic acid (chemical name: 3-[[3-(3,4-dihydroxyphenyl)-1-oxo-2-propen-1-yl]oxy]-1,4,5-trihydroxy-cyclohexanecarboxylic acid, (1S,3R,4R,5R)); Formula 4), is a cinnamate ester obtained by formal condensation of the carboxy group of trans-caffeic acid with the 3-hydroxy group of quinic acid and first isolated from green coffee beans (Freudenberg, Ber. 53, 237, 1920). This compound scavenges free radicals, which inhibits DNA damage and may protect against the induction of carcinogenesis. In addition, this agent may upregulate the expression of genes involved in the activation of the immune system and enhances activation and proliferation of cytotoxic T-lymphocytes, macrophages, and natural killer cells.

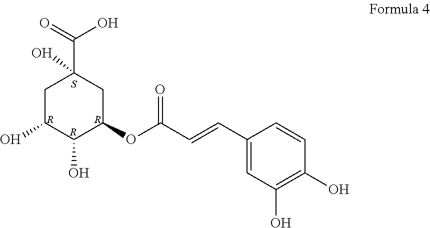

Phlorizin (chemical name: 1-[2-(D-D-glucopyranosyloxy)-4,6-dihydroxyphenyl]-3-(4-hydroxyphenyl)-1-propanone; Formula 5) is a phytochemical that belongs to the class of polyphenols. Phlorizin is a glucoside found in the stems, roots, and bark of plants in the Rosaceae family including apple, cherry, and pear. Potential and investigational uses for phlorizin include the adjuvant treatment of type 2 diabetes, as a weight loss agent for obesity, and in the acute management of hyperglycemia (Diabetes Metab Res Rev 2005; 21: 31-38).

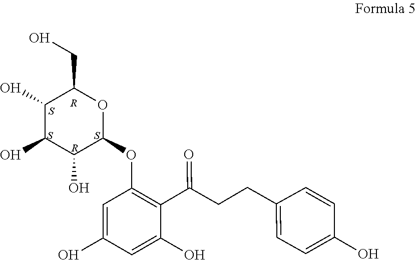

### DETAILED DESCRIPTION OF THE INVENTION

To obtain a low molecular weight HA derivative in solution conjugated with the above-mentioned bioactive agents, these compounds have been derivatized with epichlorohydrin or with 2-chloroacetic anhydride. The covalent link with low molecular weight HA did not produce a three-dimensional reticulation and the final derivative in water did not show the characteristics of a gel but the aspect of a homogeneous solution which has been used as one component for the final trimodal (or tricomponent) structuration of the final filler.

In order to obtain a crosslinking between the intermediate molecular weight HA chains the bioactive agents have been modified by introducing at least two reactive groups, epichlorohydrin or 2-chloroacetic anhydride, to favour the subsequent cross-linking reaction with the hyaluronic acid chains. In this case polydatin, gallic acid, chlorogenic acid and phlorizin covalently linked to intermediate molecular weight HA have two roles: bioactive molecules and reticulation agents.

The preparation of glycidyl polydatin derivatives is carried out by using epichlorohydrin (EP) as reagent and solvent and an organic ammonium salt as phase transfer catalysts. Specifically, tetrabutylammonium chloride (TBACl) or benzyltriethylammoniun chloride (BTEACl) were used. The followed synthetic scheme used to obtain the diglycydilated derivative of polydatin is below reported (Scheme 1).

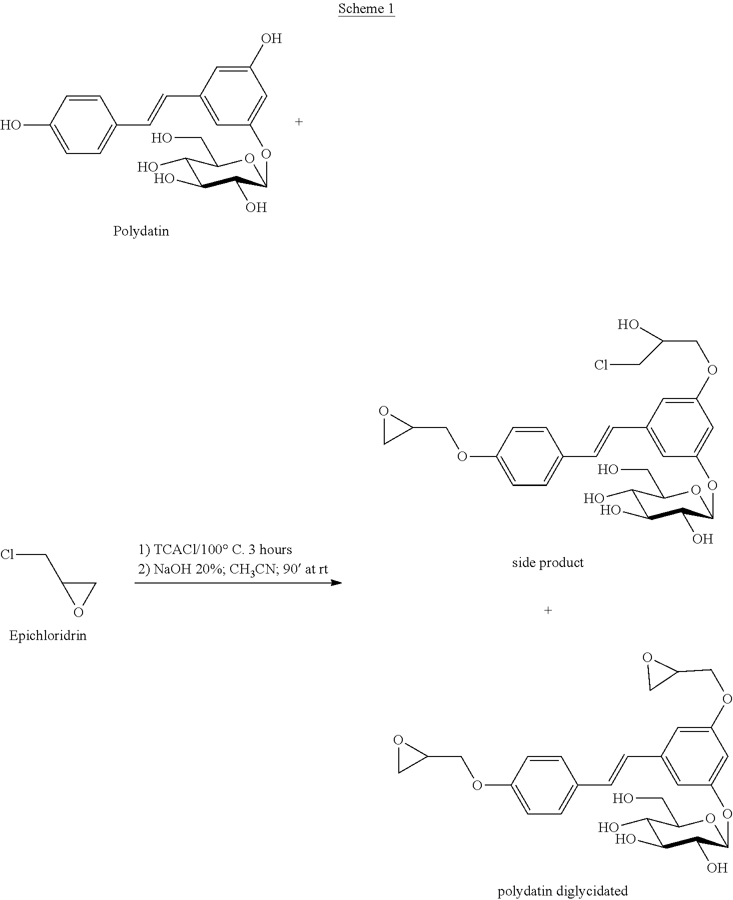

To obtain the diglycidyl derivative of polydatin (chemical name: (2R,3S,4S,5R,6S)-2-(hydroxymethyl)-6-(3-(oxiran-2-ylmethoxy)-5-((E)-4-(oxiran-2-ylmethoxy) styryl) phenoxy) tetrahydro-2H-pyran-3,4,5-triol) the following experimental conditions were utilized: the reaction proceeds at the temperature of 100° C. using 20 molar equivalents of EP and 0.1 equivalent of TBACl (or BTEACl) per mole of polydatin. Furthermore, in these conditions, a reaction time of 3 hours is enough to reach the maximum rate of conversion. After this period the reaction mixture is cooled down at room temperature then, under vigorous stirring an aprotic organic solvent, preferably di-isopropyl ether, was added to obtain a white solid which is recovered. The obtained crude of reaction could be further purified by column chromatography on silica gel to afford polydatin diglycidate.

To the best of our knowledge (2R,3S,4S,5R,6S)-2-(hydroxymethyl)-6-(3-(oxiran-2-ylmethoxy)-5-((E)-4-(oxiran-2-ylmethoxy) styryl) phenoxy) tetrahydro-2H-pyran-3,4,5-triol; polydatin diglycidate) is new. The chromatographic purification is essential to obtain a pure product since the reaction raw product contains a side product which was identified as (2S,3R,4S,5S,6R)-2-(3-(3-chloro-2-hydroxypropoxy)-5-((E)-4-(oxiran-2-ylmethoxy)styryl)phenoxy)-6-(hydroxymethyl)tetrahydro-2H-pyran-3,4,5-triol.

The preparation of the polydatin derivative with 2-chloroacetic anhydride was carried out according to the following synthetic scheme (Scheme 2).

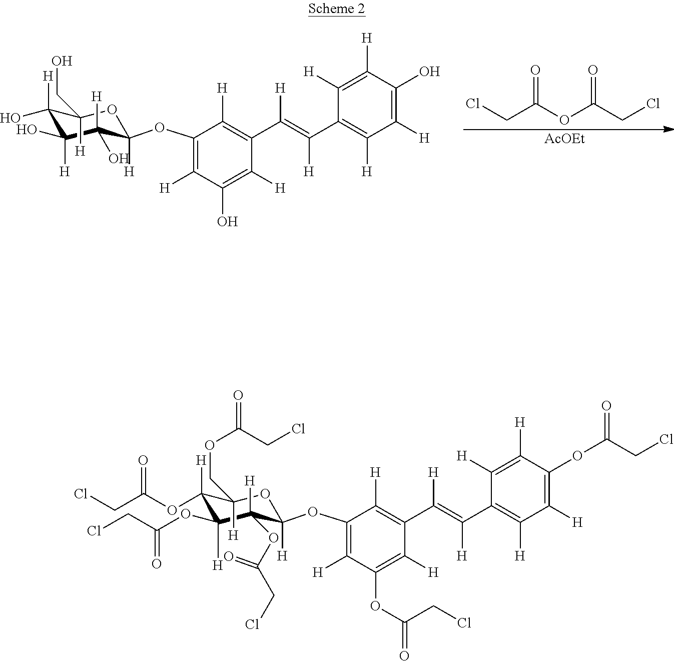

Different solvents were evaluated to obtain the 2-chloroacetylated products. The reaction in ethyl acetate (AcOEt) was found to be the most advantageous as it allows the purification of the molecule, or of the mixture of molecules, directly from the reaction mixture without the need for solvent exchange. The reaction was carried out in anhydrous conditions and under inert atmosphere (nitrogen). Polydatin (1 equivalent) was suspended in AcOEt and then, under stirring at room temperature, 6-12 equivalents of monochloroacetic anhydride were added. The reaction mixture was left at reflux for 5-10 hours, then cooled and left under stirring at room temperature for 24 hours. Then the reaction mixture was diluted with water to give a white solid precipitate recovered by suction, washed with water and dried under vacuum at room temperature affording the hexa-chloroacetyl derivative of polydatin in 91% yield. To the best of our knowledge this compound is new. A mixture of mono-, di-, tri- and tetra-2-chloroacetyl polydatin esters is 17±3.4%/44.3±8.8%/17.7±3.6%/1.9±0.4% may be obtained.

The reaction of gallic acid with 2-chloroacetic anhydride to afford the corresponding 3,4,5-tris(2-chloroacetoxy) benzoic acid is reported in the following scheme (Scheme 3).

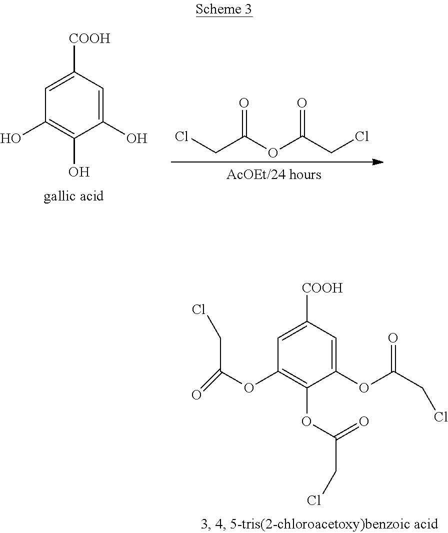 [SMILES: O=C(O)c1cc(O)c(O)c(O)c1 ; O=C(CCl)OC(=O)CCl ; O=C(CCl)Oc1cc(C(=O)O)cc(OC(=O)CCl)c1OC(=O)CCl]

The general synthetic procedure comprises the reaction between gallic acid and 4-8 milliequivalents of 2-chloroacetic anhydride in ethyl acetate, preferably 6 milliequivalents. The reaction temperature ranges between 1° and 30° C. preferably 20-25° C. The reaction is usually completed in about 24 hours. The organic phase is first washed with an acidic water solution, preferably HCl 0.5M, then with brine and subsequently dried on Na₂SO₄, filtered and the filtrate evaporated under vacuum to afford an oily residue. —This oil is then treated with water to afford a white solid which is then dried under vacuum. The overall molar yield is 79%. To the best of our knowledge this gallic acid derivative is new.

The reaction with gallic acid and epichlorohydrin is carried out according to the following synthetic scheme (Scheme 4).

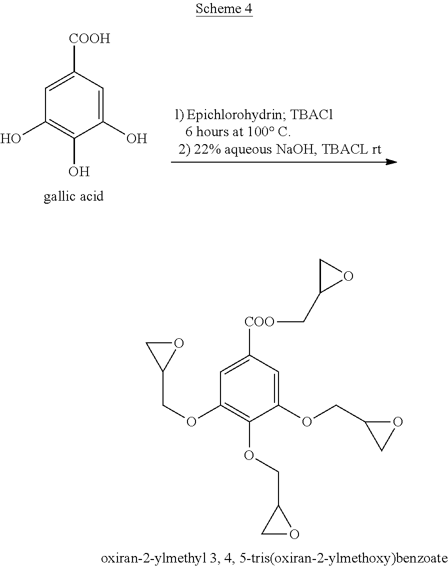

The preparation of the glycidyl derivative was carried out by mixing gallic acid (GA) and EP in the presence of an organic salt (tetrabutylammonium chloride, TBACl), as a phase transfer catalyst. In this reaction EP acts as both a reagent and a solvent. The relative molar ratio between GA and EP is comprised in a range from 1/14 to 1/18, preferably 1/16. The procedure involves the sequential addition of GA, TBACl and EP, subsequently leaving the mixture for 6 hours in a temperature range ranging between 6° and 100° C., preferably 100° C. The reaction mixture was then cooled to room temperature and treated with 20% w/w NaOH solution (2 molar equiv./OH) and 0.1 molar equivalents of TBACl. The resulting white suspension is shaken vigorously at room temperature and then the reaction mixture iss diluted four times with water extracted three times with AcOEt. The combined organic phases are washed with a saturated NaCl solution dehydrated with anhydrous Na₂SO₄ and evaporated at reduced pressure to afford a crude which can be further purified by chromatography to afford the desired product

Similarly, glycidyl and 2-chloroacetyl derivatives of chlorogenic acid and phlorizin were obtained.

The preparation of functional matrices by conjugating the polydatin derivatives above mentioned with hyaluronic acid of intermediate molecular weight was then carried out.

The critical parameters for the preparation of these new hyaluronic acid derivatives are reported below. Depending on the type of solvent of reaction utilized, on the reaction temperature and time and on the relative molar ratio between the activated molecules (ie polydatin, gallic acid, chlorogenic acid and phlorizin derivatized with epichlorohydrin and 2-chloroacetic acid) and hyaluronic acid different characteristic of viscosity, elasticity and stability (thermal and enzymatic) were obtained.

Type of solvents. Since the derivatization of the ancillary molecules (polydatin, gallic acid chlorogenic acid and phlorizin) determines a significant decrease in their water solubility, it was necessary to use an organic solvent for their solubilization. In our experiments the solvent used was dimethyl sulfoxide (DMSO) a solvent endowed with low toxicity, polar and water soluble. On the other hand, hyaluronic acid, in its sodium salt form, is completely soluble in water and demonstrates a modest and limited solubility in DMSO. On the basis of these considerations, different reaction conditions were evaluated based on the use of DMSO, H₂O and DMSO/H₂O mixtures. Experimental evidence indicates that the use of water alone does not promote the conjugation reaction. The use of DMSO as unique reaction solvent allows the reaction to proceed by generating conjugates which, once solubilized in water, form solutions.

We surprisingly found that the use of a DMSO/H₂O mixture, depending on the percentage of H₂O used, favours the preparation of both a water-soluble conjugate and of a true gel. In particular, the use of the mixture of solvents in a 1:1 ratio favours the formation of gels.

Temperature of reaction. The reaction temperature directs the degree of substitution on the polysaccharide chain. The range considered is included in the range 30-80° C. The lower limit of this interval does not produce conjugation while at 50° C. the reaction begins to take place. The temperature of 50° C. has been chosen as the best temperature for carrying out the reactions since, at this temperature, there is evidence of conjugation and gel formation. While introducing possible alterations of the structure, working at the upper limit of 80° C. produces conjugated structures that also have properties of gels.

Time of reaction. During the early stages of the reaction conditions setup, in-process controls (IPCs) were conducted to assess the progress of the reaction under the conditions previously described. The kinetics were rather slow and the first evidence of conjugation occurs after 3 hours with an appreciable substitution around 15 hours. The use of the mixture of solvents in 1:1 ratio, as mentioned above, allowed to speed up the kinetic process within 2 hours.

Molar ratio between derivatized ancillary molecules and hyaluronic acid. The quantities used in the reactions and in particular the molar ratios between them regulate the degree of substitution on the polymer chain. The ratios used were 1/1, 1/5 and 1/10, expressed between moles of derivatized ancillary molecule (for example the polydatin derivatives) and moles of the dimeric unit constituting the hyaluronic acid chains. We found that the 1/5 and 1/1 ratio increase the load of the conjugated ancillary molecule.

The reaction conditions utilized for the preparation of gels using hyaluronic acid of three different molecular weight (HMW, MMW and LMW) with diglycidylate polydatin (PO_{DG}) and characterization are discussed below.

These reaction conditions were applied, with minor changes, on all the glycidyl derivatives of the ancillary molecules of the invention (i.e. gallic acid, chlorogenic acid and phlorizin).

### Cross-Linking Degree

A cross linking degree is preferably selected so as to give a ratio of viscous modulus (G″) to elastic modulus (G′) of less than 1.0. When 2-chloroacetylated polydatin is used as cross-linker, in a molar ratio of chlororoacetilated polydatin 2—with respect to the moles of repeating units of HA comprised between 1:5 to 1:10, the obtained degree of crosslinking is comprised between 70 and 80%. When diglycidated polydatin is used as cross-linker, in a molar ratio of diglycidated polydatin with respect to the moles of repeating units of HA comprised between 1:5 to 1:1, the obtained degree of crosslinking is comprised between 15 and 55%.

The following critical reaction parameters relevant for the crosslinking reaction were studied:
relative molar ratios between PO_{DG} and hyaluronic acid; the following molecular ratios were investigated: 5/1; 1/1; 1/5; 1/10.
three fractions of hyaluronic acids were tested: HMW, MMW, LMW
were considered the reaction time of 1 hour and 4 hours, preferably 2 hours

Other process parameters were kept constant: the reaction temperature was always 50° C. and the solvent used was a H₂O/DMSO mixture in a constant 1:3 or 1:1 v/v ratio.

A preliminary general crosslinking procedure involves the use of an aqueous 0.25M NaOH solution in which hyaluronic acid of a defined molecular weight is solubilized at room temperature (the balance between the use of a basic solution and the temperature is relevant in order not to degrade hyaluronic acid). Therefore, during the preparation phase, the hyaluronic acid was conditioned in a basic environment at room temperature for a variable period of time (depending on the molecular weight from 1 hour for HMW to 30 min for LMW).

In parallel, a solution of PO_{DG} in DMSO was prepared. The derivatization of polydatin with epoxy groups leads to a significant reduction in the solubility in water, which in itself is quite low. It was therefore necessary to use DMSO as reaction co-solvent. Once both components (hyaluronic acid and PO_{DG}) were dissolved, the DMSO solution is poured onto the hyaluronic acid solution in 0.25M NaOH and the temperature is increased to 50° C. The reaction is maintained under stirring for 1 hour or 4 hours. At the end of the reaction, it is necessary to precipitate the polymer to eliminate the excess reagents. For crosslinked with PO_{DG}, the workup of the reaction involves the addition of a low molecular weight alcohol, preferably ethanol. In these experimental conditions, the polymer appears as a sediment and can be isolated by centrifugation. The final step involves the hydration of the precipitate in deionized water (MilliQ water), prior to the purification of the salts still present by dialysis. The final isolation of the hyaluronic acid derivative is then done by lyophilization.

The use of HA with MMW and LMW in the same the reaction conditions confirmed that for obtaining gel with PO_{DG} the optimum PO_{DG}/HA ratio is of 1/1, the reaction temperature is 50° C. and a reaction time comprised between 1 and 4 hours.

Preliminary tests carried out on heat sterilization of the obtained gel samples gave further indications relevant for the development of a stable sterile gel formulation. In fact, the gels produced with poly 2-chloro acetylated polydatin blends do not withstand thermal stress. This treatment generates, at the end of the sterilization cycle, a solution and no longer a gel. The process induces a deconstruction in these gels, probably due to the hydrolysis of the ester bonds, which cause crosslinking, and which are particularly labile to heat. On the other hand, the gels produced with diglycidylated polydatin (PO_{DG}) are not affected by the thermal stress applied in such an extensive manner, as the ether bonds, which cause cross-linking, are less susceptible to thermal degradation.

On the basis of these sterilization results gel preparations were developed based exclusively on glycidylated crosslinkers for moist heat sterilization whereas 2-chloroactylated ancillary crosslinkers were preferred for gamma rays sterilization.

The cross-linked hyaluronic acid of the invention are useful in dermal filler compositions in an amount of between 1 mg/ml and 50 mg/ml optionally in the presence of an anaesthetic, preferably lidocaine at a final concentration comprised between 0.1 and 0.4% weight/volume. The compositions will be used in methods for replacing or filling of a biological tissue or increasing the volume of a biological tissue for cosmetic purposes.

The injectable compositions of the invention are administered Intradermally or intraarticularly in the form of sterile gels. A mixture of cross-linked hyaluronic acids of different molecular weights may be used.

The compositions may be presented in form of kits comprising instructions for use and possibly other useful agents.

The invention is detailed in the following Examples.

### Example 1—Preparation of polydatin diglycidylate (2R,3S,4S,5R,6S)-2-(hydroxymethyl)-6-(3-(oxiran-2-ylmethoxy)-5-((E)-4-(oxiran-2-ylmethoxy) styryl) phenoxy) tetrahydro-2H-pyran-3,4,5-triol)

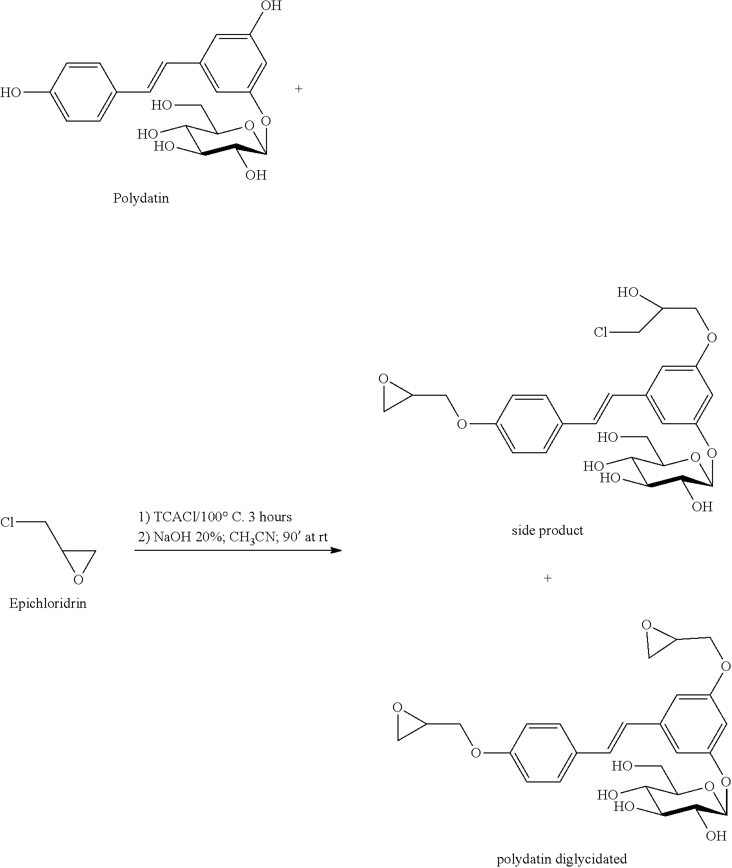

The reaction was carried out under an inert atmosphere (Argon). 58 mg BTEACl (0.256 mmol, 0.1 molar equiv.) and 4 mL epichlorohydrin (51.2 mmol, 20 molar equiv.) were added to 1.0 g polydatin (2.56 mmol). The resulting suspension was heated to 100° C. under stirring. After 30 min at 100° C. the suspension became a clear straw-coloured solution, heating was continued for 3 hours and allowed to cool to room temperature. Before the reaction mixture solidifies, 40 mL of di-isopropyl ether were added under vigorous stirring. A white solid was immediately separated and filtered by washing with 5 mL of the same solvent. The solid obtained (1.18 g) by TLC analysis (CH2Cl2/MeOH 90/10) consists of two compounds.

The crude solid obtained was dissolved in 20 mL of MeOH (with slight heating), 7 g of SiO₂ was added to the solution and the solvent was evaporated in a rotavapor. The powder obtained, after complete removal of the MeOH by mechanical vacuum pumping, was loaded into a SiO₂ flash chromatography column that was eluted with CH₂Cl₂/MeOH 90/10 v/v. The first eluted product is the desired diglycidylated derivative of polydatin 3, 449 mg (35% molar yield), which was crystallised from 25 mL of ethanol. The compound had m.p. 167-170° C., [α]_{D}25° C.=−50° (c 0.5 in MeOH) and [α]_{D}25° C.=−36° (c 1 in DMSO). The compound was subjected to NMR analysis (¹H, ¹³C, H,H COSY, ETCORR), which enabled all signals to be assigned, confirming the structure. The numeration refers to the formula reported in formula 6.

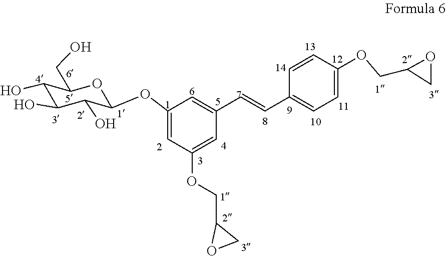 [SMILES: OCC1OC(Oc2cc(/C=C/c3ccc(OCC4CO4)cc3)cc(OCC3CO3)c2)C(O)C(O)C1O]

¹H NMR (500 MHz, DMSOd6): δ 7.51 (2H, d, J=8.8 Hz, H-10 e 14), 7.19 (1H, d, J=16.4 Hz, H-8), 7.00 (1H, J=16.4 Hz, H-7), 6.98 (2H, d, J=8.8 Hz, H-11 e 13), 6.88 (1H, br s, H-6), 6.82 (1H, br s, H-4), 6:55 (1H, m, H-2), 5.31 (1H, d, J=4.9 Hz, OH on C-2′), 5.12 (1H, d, J=4.4, OH on C-3′), 5.05 (1H, d, J=5.2 Hz, OH on C-4′), 4.87 (1H, d, J=7.0 Hz, H-1′), 4.67 (1H, dd, J=5.1 e 5.1 Hz, OH on C-6′), 4.36 (2H, dd, J=11.4 e 2.6 Hz, H-1a″×2), 3.84 (2H, dd, J=11.4 e 6.6 Hz, H-1b″×2), 3.74 (1H, dd, J=10.4 e 5.2 Hz, H6a′), 3.46 (1H, m, H-6b′), 3.40-3.32 (3H, overlapping, H-5′ e H-2″×2), 3.31-3.22 (2H, overlapping, H-2′ e H-3′) 3.15 (1H, dd, J=8.8 e 5.1 Hz, H-4′), 2.85 (2H, m, H-3a″×2), 2.71 (2H, m, H-3b″×2); ¹³C NMR (125 MHz, DMSOd6): δ 159.3 (C-12), 158.7 (C-1), 157.9 (C-3), 139.3 (C-5), 129.8 (C-9), 128.6 (C-8), 127.8 (C-10 e C-14), 126.0 (C-7), 114.7 (C-11 e C-13), 106.9 (C-6), 106.1 (C-4), 102.0 (C-2), 100.5 (C-1′), 77.1 (C-5′), 76.7 (C-3′), 73.2 (C-2′), 69.8 (C-4′), 68.9 (C-1″×2), 60.7 (C-6′), 49.6 (C-2″×2), 43.7 (C-3″×2). The Ms spectrometry (ESI) shows a peak at m/z=525.8, which corresponds to the addition of Na⁺ to the molecular ion.

The second product eluted was a side product, (261 mg) which was crystallised from 15 mL of isopropyl alcohol to give a solid showing m.p. 138-141° C. (dec.), [α]_{D}25° C.=−43° (c 1 in MeOH).

The compound was subjected to NMR analysis (¹H, ¹³C, H,H COSY, ETCORR) and was found to consist of a mixture of two very similar (and non-chromatographically separable) compounds in a ratio of about 3:1, the two compounds differing in the position of the glycidyl and chloroalcohol substituents. NMR data of the main component 4A are reported below. The numbering system is reported in formula 7 (chemical name: (2S,3R,4S,5S,6R)-2-(3-(3-chloro-2-hydroxypropoxy)-5-((E)-4-(oxiran-2-ylmethoxy)styryl)phenoxy)-6-(hydroxymethyl) tetrahydro-2H-pyran-3,4,5-triol).

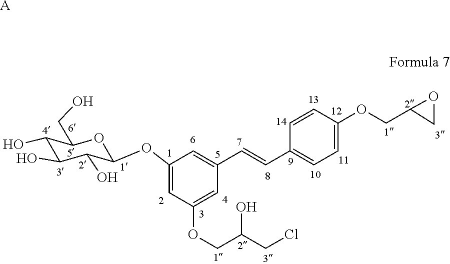

¹H NMR (500 MHz, DMSOd6): δ 7.53 (2H, d, J=8.5 Hz, H-10 e 13), 7.19 (1H, d, J=16.4 Hz, H-8), 7.02 (1H, d, J=16.4 Hz, H-7), 6.98 (2H, d, J=8.5 Hz, H-11 e 13), 6.89 (1H, br s, H-6), 6.81 (1H, br s, H-4), 6.52 (1H, br s, H-2), 5.58 (1H, d, J=4.0 Hz, OH in 3′″), 5.30 (1H, d, J=4.7 Hz, OH in 2′), 5.11 (1H, d, J=4.0 Hz, OH in 3′), 5.05 (1H, d, J=4.9 Hz, OH in 4′), 4.88 (1H, d, J=7.3 Hz, H-1′), 4.67 (1H, dd, J=5.0 e 5.0 Hz), 4.35 (1H, dd, J=11.4 e 2.1 Hz, H-1″a), 4.03 (1H, m, H-2′″), 4.01 (2H, m, H-1′″a e 1′″b), 3.85 (1H, dd, J=11.4 e 6.5 Hz, H″b), 3.75 (1H, dd, J=11.1 e 4.4 Hz, H-3′″a), 3.73 (1H, m, H-6′a), 3.68 (1H, dd, J=11.1 e 5.1 Hz, H-3′″b), 3.47 (1H, dd, J=11.7 e 5.9 Hz, H-6′b), 3.38-3.33 (2H, overlapping, H-2″ e 5′), 3.28 (1H, m, H-3′), 3.26 (1H, m, H-2′), 3.15 (1H, m, H-4′), 2.85 (1H, m. H-3″a), 2.71 (1H, m, H-3″b); ¹³C NMR (125 MHz, DMSOd6): δ 159.5 (C-3), 158.7 (C-1), 157.9 (C-12), 139.3 (C-%), 129.8 (C-9), 128.6 (C-8), 127.8 (2C, C-10 e 14), 126.0 (C-7), 114.7 (2C, C-11 e 13), 106.8 (C-6), 106.2 (C4), 102.1 (C-2), 100.6 (C-1′), 77.1 (C-5′), 76.6 (C-3′), 73.2 (C-2′), 69.8 (C-4′), 69.1 (C-1′″), 68.9 (C-1″), 68.6 (C-2′″), 60.7 (C-6′), 49.6 (C-2″), 46.7 (C-3′″), 43.7 (C-3″).

### Example 2—Preparation of polydatin hexa-2-chloroacetyl derivative

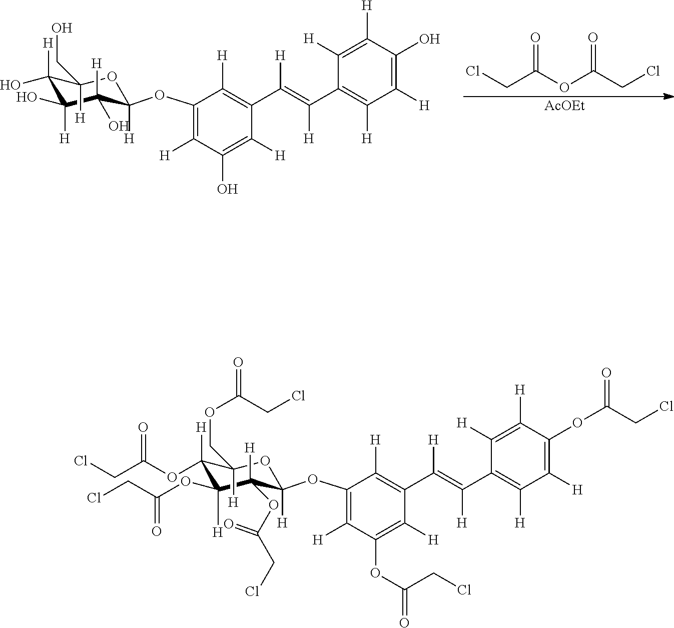

The reaction was carried out in anhydrous conditions and under inert atmosphere (nitrogen). Polydatin (2.0 g, 5.13 mmol) was suspended in AcOEt (16 ml) and then, under stirring at room temperature, monochloroacetic anhydride (8.7 g, 51.2 mmol) was added. The reaction was brought to reflux and after 45 min solubilisation was observed. The reaction mixture was left for a further 6 hours 15 min at reflux, then cooled and left under stirring at room temperature for 24 hours. Then 20 mL of H₂O were added and the obtained mixture stirred for 30 min. The formation of a white solid precipitate was observed. The solid was filtered by suction and washed with water (20 mL×3). The white solid was dried under vacuum at room temperature for 48 hours affording 4.3 g of product (91% yield).

Characterization: [α]_{D}²⁵=−12.00 (c=1.0; CHCl₃); ¹H NMR (500 MHz, CDCl₃): δ=7.51 (overlapping, 2H, H-10 and H-14), 7.16 (overlapping, 2H, H-11 and H-13), 7.07 (d, J_{7,8}=16.2 Hz, 1H, H-7), 7.04 (s, 1H, H-6), 7.01 (s, 1H, H-8), 6.97 (d, J_{7,8}=16.2 Hz, 1H, H-8), 6.78 (s, 1H, H-2), 5.43 (t, J=9.3 Hz, 1H, H-3′), 5.37 (t, J=9.3 Hz, 1H, H-2′), 5.25 (t app, J=9.3 Hz, 1H, H-4′), 5.20 (d, J=7.6 Hz, 1H, H-1′), 4.39-4.35 (overlapping, 2H; H-6a′ and H-6b′), 4.33-4.29 (overlapping, 4H, 2×CH₂Cl), 4.10 (s, 2H, CH₂Cl), 4.07 (s, 2H, CH₂Cl), 4.06-4.00 (overlapping, 5H, 2×CH₂Cl and H-5); ¹³C NMR (125 MHz, CDCl₃): δ=166.9 (2C, CO at C6′ and CO at C3′), 166.2 (CO at C4′), 160.0 (CO at C2′), 165.8 and 165.7 (2C, 2×CO at Ph), 157.1 (C1), 151.3 (C3), 150.1 (C12), 140.0 (C5), 134.7 (C9), 129.9 (C7 or C8) 127.8 (2C, C10 and C14), 127.3 (C7 or C8), 121.5 (2C, C11 and C13), 114.3 (C6), 113.6 (C4), 109.2 (C2), 98.5 (C1′), 73.6 (C3′), 72.1 (C2′), 71.6 (C5′), 69.5 (C4′), 63.2 (C6′), 40.8 (2C, CH₂Cl), 40.5 (CH₂Cl), 40.3 (CH₂Cl), 40.2 (CH₂Cl), 40.1 (CH₂Cl). MS (ESI positive): m/z: 851.0 [M+H]⁺.

### Example 3—Preparation of 3,4,5-tris(2-chloroacetoxy)benzoic acid from gallic acid

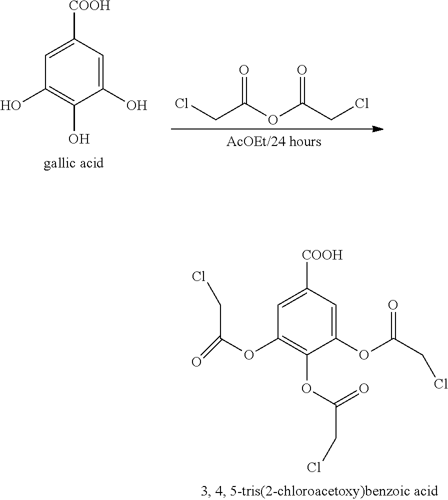 [SMILES: O=C(O)c1cc(O)c(O)c(O)c1 ; O=C(CCl)OC(=O)CCl ; O=C(CCl)Oc1cc(C(=O)O)cc(OC(=O)CCl)c1OC(=O)CCl]

2-chloroacetic anhydride (3 g; 17.6 mmoles) was added to a suspension of gallic acid (0.5 g; 2.94 mmoles) in ethyl acetate (3 ml) under stirring at room temperature. The reaction proceeded at room temperature and was completed after 24 h. The reaction mixture was then treated with aqueous HCl 0.5M (6 ml) and stirred for 0.5 hours to decompose the excess of anhydride. The organic phase was separated and washed with brine (3 times). The organic phases were dried on Na₂SO₄, filtered and the filtrate evaporated under vacuum to afford an oily residue. This oil was then treated with water to afford a white solid which was dried under vacuum (25° C. for 10 h and 50° C. for 3 hours) to afford 0.925 g of product as white solid (2.3 mmoles; 79% molar yields). Physicochemical properties: m.p. 158-159° C.; ¹H NMR (500 MHz, DMSO-d₆): δ 7.92 (overlapping, 2H, C3 and C7), 4.83 (s, 2H, CH₂Cl), 4.77-4.73 (overlapping, 4H, 2×CH₂Cl); 13C NMR (125 MHz, DMSO-d₆): δ 165.2 (2C, COCH₂Cl at C4 and C6), 164.9 (C1), 164.3 (COCH₂Cl at C5), 142.6 (2C, C4 and C6), 137.2 (C5), 129.7 (C2), 122.3 (C3 and C7), 40.9 (2C, COCH₂Cl at C4 and C6), 40.3 (COCH₂Cl at C5). Mass spectroscopy confirmed the molecular weight corresponding to C₁₃H₉Cl₃O₈. MS (ESI negative) [M−H]⁻ m/z: 396.6 (100%), 398.7 (100%), 400.3 (45%).

### Example 4—Preparation of oxiran-2-ylmethyl 3,4,5-tris(oxiran-2-ylmethoxy) benzoate from gallic acid

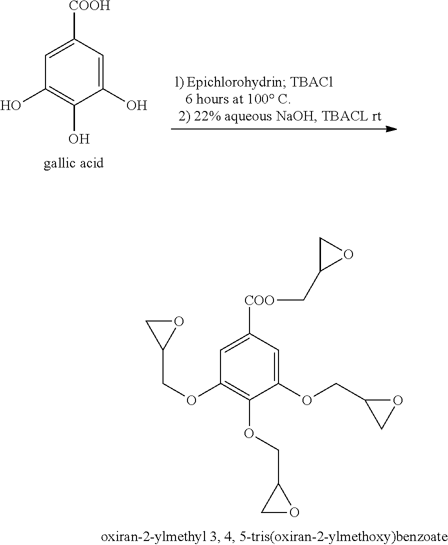

The reaction was carried out under inert atmosphere (Argon) to avoid possible oxidation of gallic acid. To a mixture of 2.0 g (11.76 mmol) of gallic acid and TBACl (266 mg, 1.18 mmol, 0.1 molar equiv.), 14.68 mL of epichlorohydrin (187.6 mmol, molar ratio gallic acid/epichlorohydrin 1:16) were added and the resulting white suspension was stirred at 100° C. After 30 min, a straw-coloured solution was obtained and stirring continued at 100° C. for 6 hours. After cooling to room temperature, 15.4 mL of 20% w/w NaOH solution (2 molar equiv./OH) and 266 mg TBACl were added. The resulting white suspension was shaken vigorously at room temperature for 90 min. 60 mL of water were then added and extracted with AcOEt (3×60 mL). The combined organic phases were washed with a saturated NaCl solution (2×80 mL), dehydrated with anhydrous Na₂SO₄ and evaporated at 70° C. under reduced pressure to give 2.50 g crude as a straw oil. This crude was purified by chromatography (flash chromatography): by elution with petroleum ether/AcOEt 20/80, the desired product (1.01 g; 22% yield) was obtained. ¹³C-NMR spectroscopy data on the obtained sample are in agreement with published literature data (Aouf, Chahinez; Tetrahedron 2013, 69(4), 1345-1353).

### Example 5—Preparation of the hepta-chloroacetyl derivative of phlorizin

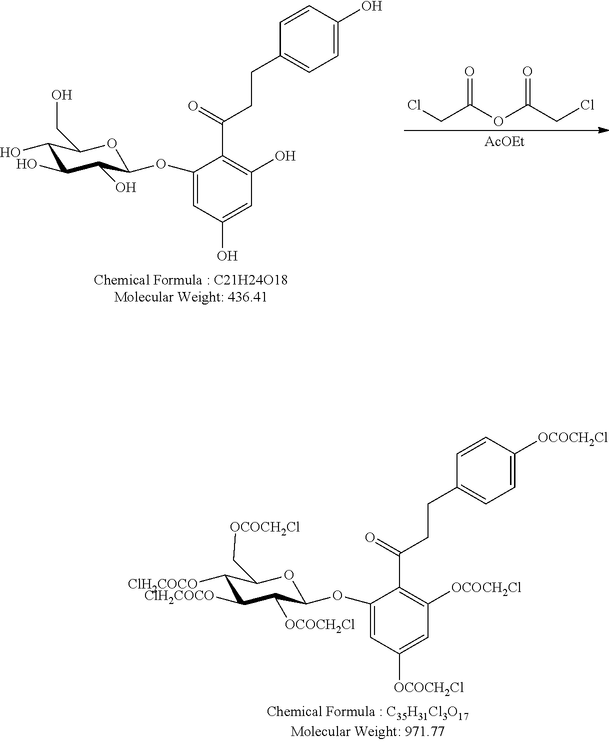

The system is dried under argon, phlorizin (200 mg, 0.51 mmol) and monochloroacetic anhydride (0.7 g, 4.09 mmol) were suspended in AcOEt, the reaction was brought to reflux (80° C.), to give after a few minutes a solution which was left under stirring at reflux for 3 h and overnight at room temperature. After this period monochloroacetic anhydride (0.3 g, 1.75 mmol) was added and the reaction was refluxed for 4 hr. 6 mL of 0.5 M HCl were added and stirred for 30 minutes. The organic phase was extracted 3 times with AcOEt (10 mL×3); the combined organic phases were washed with saturated NaCl solution (brine, 12 mL). The organic phase was dried over Na₂SO₄ and concentrated under vacuum. 6 mL of H₂O were added to the oily residue and left on ice, and after 20 minutes a further 6 mL of water were added. After a further washing with NaHCO₃ and then with brine the organic phases were dried over sodium sulphate and concentrated under vacuum. The desired product was obtained as a white solid residue (345 mg, 79%) m.p. 145-148° C.; [α]_{D}²⁵=−19.00 (c=1.0; CHCl₃); ¹H NMR (500 MHz, CDCl₃): δ=7.27 (overlapping, 2H, H-11 and H-14), 7.05 (overlapping, 2H, H-12 and H-14), 6.96 (d, J_{2,4}=1.9 Hz, 1H, H-2), 6.82 (d, J_{4,2}=1.9 Hz, 1H, H-4), 5.39 (t app, J_{3′,2′}=9.4 Hz, 1H, H-3′), 5.31 (dd, J_{2′,1′}=7.8, J_{2′,3′}=9.4 Hz, 1H, H-2′), 5.17 (t app, J=9.6 Hz, 1H, H-4′), 5.07 (d, J=7.8 Hz, 1H, H-1′), 4.35 (dd, J_{3′,2′}=2.5, J_{6a′,6b′}=12.4 Hz, 1H, H-6a′), 4.33-4.28 (overlapping, 5H, 2×CH₂C and H-6b′), 4.14 (s, 2H, CH₂Cl), 4.05-4.00 (overlapping, 5H, 2×CH₂C and H-5), 4.04 (s, 2H, CH₂Cl), 3.98 (s, 2H, CH₂Cl), 3.13-3.04 (m, 2H, H8a and H8b), 3.04-2.87 (m, 2H, H9a and H9b); ¹³C NMR (125 MHz, CDCl₃): δ=199.9 (C7), 166.9, 166.7 (2C, CO at C6′ and CO at C3′), 166.2, 160.0 (2C, CO at C4′ and CO at C2′), 165.1 and 165.0 (2C, 2×CO at Ph), 154.0 (C1), 151.5 (C3), 148.6 (C13), 147.3 (C5), 139.0 (C10), 129.8 (2C, C11 and C15), 123.7 (C6), 121.1 (2C, C12 and C14), 111.5 (C4), 107.7 (C2), 99.3 (C1′), 73.1 (C3′), 71.9 (C5′), 71.3 (C2′), 69.3 (C4′), 63.2 (C6′), 46.0 (C8), 40.9 (CH₂Cl), 40.7 (CH₂Cl), 40.5 (CH₂Cl), 40.3 (CH₂Cl), 40.2 (2C, CH₂Cl), 40.1 (CH₂Cl), 28.4 (C9); MS (ESI positive): m/z 973.1 [M+H]⁺.

### Example 6—Preparation of the penta(chloroacetyl) derivative of chlorogenic acid

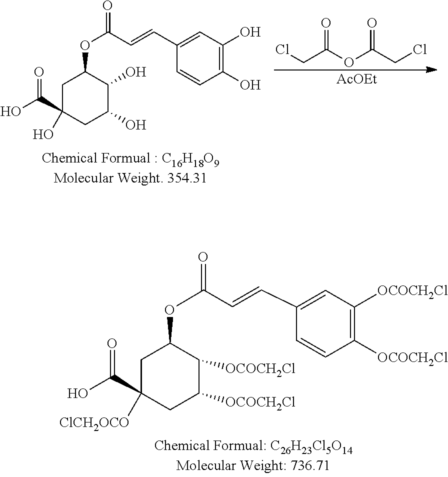

The system was dried under argon, chlorogenic acid (200 mg, 0.56 mmol) and monochloroacetic anhydride (0.677 g, 3.96 mmol) were suspended in AcOEt, the reaction was brought to reflux (bath 80° C.), to give a solution after 15 min. The mixture was left under stirring for 6 hours then mL of 0.5 M HCl were added 6 and the mixture stirred for additional 30 min. The reaction mixture was extracted 3 times with AcOEt (10 mL×3); the pooled organic phases were washed with saturated NaCl solution (12 mL). The organic phase was dried over Na₂SO₄ and concentrated under vacuum. 6 mL of H₂O were added to the oily residue, cooled to 0° C. to afford the desired product as a powder (224 mg, 40% yield). m.p. 118-122° C.; [α]_{D}²⁵=−21.00 (c=1.0; CHCl₃); ¹H NMR (500 MHz, CDCl₃): δ=7.81 (d, J_{5′,9′}=1.8 Hz, 1H, H-5′), 7.74 (dd, J_{9′,5′}=1.8, J_{9′,8′}=8.8 Hz, 1H, H-9′), 7.63 (d, J_{3′,2′}=16.0 Hz, 1H, H-3′), 7.43 (d, J_{8′,9′}=8.8 Hz, 1H, H-8′), 6.62 (d, J_{2′,3′}=16.0 Hz, 1H, H-2′), 5.55-5.52 (m, 1H, H-6), 5.42-5.36 (overlapping, 2H, H-4 and H-5), 7.78-7.71 (overlapping, 4H, 2×CH₂Cl), 4.60-4.23 (overlapping, 6H, 3×CH₂Cl), 2.62 (dd, J_{7a,6}=3.4, J_{7a,7b}=16.0 Hz, 1H, H-7a), 2.56-2.51 (overlapping, 2H, H-3a and H-7b), 2.19 (dd, J_{3b,4}=9.9, J_{3b,3a}=12.9 Hz, 1H, H-3b); ¹³C NMR (125 MHz, CDCl₃): δ=170.3 (C1), 166.9, 166.4, 166.1, 165.4, 165.3, 164.8 (6C, C1′, CO at C2, CO at C6, CO at C5, CO at C6′, CO at C7′), 143.2 (C3′), 142.7 (C7′), 141.6 (C6′), 133.2 (4′), 127.7 (9′), 124.0 (8′), 123.0 (5′), 118.9 (C2′), 80.0 (2), 72.1 (C4), 69.5 (C6), 66.5 (C5), 41.4 (CH₂C), 40.9 (CH₂Cl), 40.8 (CH₂Cl), 40.7 (2C, 2×CH₂Cl), 35.8 (C3), 31.3 (C7); MS (ESI negative): m/z 734.9 (100%) [M−H]⁻, 737.0 (80%) [M−H]⁻, 733.1 (65%) [M−H]⁻.

General Procedure for the Crosslinking Using Glycidated Compounds: Hyaluronic Acid Cross-Linked with Diglycidated Polydatin

100 mg of hyaluronic acid sodium salt (HANa) is added to 2 or 4 mL (Col A Tab1) of 0.25 M NaOH and the mixture is vortexed and left for 15 min at r.t. A solution of 0, 25, 50 or 78.2 mg (Col C Tab1) of polydatin diglycidylate (PO_{DG}) dissolved in 1 or 2 mL (Col D) of DMSO is then added. The PO PO_{DG}:HANa (repeating unit) mole ratios are given in Col F. The mixture is heated under stirring at 50° C. for 2 hours. It is then neutralised (pH ca 7) with 1 M HCl (approximately 0.95 mL in the case of 4 mL of 0.25 M NaOH and 0.4 mL in the case of 2 mL of 0.25 M NaOH). 5 or 10 mL of EtOH (Col G) are added to precipitate the polymer, vortexed for 2 min and centrifuged at 4000 g for 10 min. The supernatant (surn1) is analysed in the UV spectrophotometer to determine the mg of PO_{DG} present (unbound to hyaluronic acid and by difference the proportion of PO_{DG} cross-linked with hyaluronic acid; Table 1).

TABLE 1
Reaction conditions

| | | | | | | | | | Col H | Col I | Col L | Col M |
|---|---|---|---|---|---|---|---|---|---|---|---|---|
| | | Col A | Col B | Col C | Col D | Col E | | Col G | washing 2 | washing 3 | Weight after | POdg unbounded |
| | Test | mL NaOH | Concentr. | mg | mL | % cross- | Col F | washing 1 | mL EtOH/ | and next | liophilization | recovered from |
| Entry | number | 0.25M | mg/mL | PO_{DG} | DMSO | linker^{a} | ratio^{b} | mL EtOH | H₂O 4:1 | mL EtOH | mg | the washings |
| 1 | PR006* | 4 | 25 | 25 | 1 | 20 | 1:5 | 10 | 5 | 4 | 90.7 | 100% |
| 2 | PR006A* | 4 | 25 | 0 | 1 | 0 | | 10 | 5 | 4 | 111.2 | — |
| 3 | PR007* | 2 | 50 | 25 | 1 | 20 | 1:5 | 5 | 5 | 4 | 106.5 | 71% |
| 4 | PR007A* | 2 | 50 | 0 | 1 | 0 | | 5 | 5 | 4 | 118.1 | — |
| 5 | PR008* | 2 | 50 | 25 | 1 | 20 | 1:5 | 5 | 5 | 4 | 91.4 | 66% |
| 6 | PR009* | 2 | 50 | 25 | 1 | 20 | 1:5 | 2 H₂O + | 5 | 4 | 111.6 | 63% |
| | | | | | | | | 10 EtOH | | | | |
| 7 | PR010* | 4 | 25 | 25 | 1 | 20 | 1:5 | 10 | 5 | 4 | 109.8 | 95% |
| 8 | PR011* | 2 | 50 | 25 | 1 | 20 | 1:5 | 5 acetone | 5 acetone/ | 4 acetone | | |
| | | | | | | | | | H₂O 4:1 | | | |
| 9 | PR012* | 2 | 50 | 50 | 2 | 40 | 1:2.5 | 5 | 5 | 4 | | 63% |
| 10 | PR013* | 2 | 50 | 78.2 | | 62.5 | 1:1.6 | 5 | 5 | 4 | | 67% |
| 11 | PRO15* | 2 | 50 | 125 | 2 | 100 | 1:1 | 5 | 5 | 4 | 150 | 55% |
| 12 | PRO19** | 2 | 50 | 125 | 2 | 100 | 1:1 | 5 | 5 | 4 | 100 | 80% |

^{a}% cross-linker = mol PO_{DG}/mol HA (ripetitive unit) × 100
^{b}ratio = mol PO_{DG}:mol HA (ripetitive unit)
*Utilized Medium MW Hyaluronic acid (500-750 KDalton)
**Utilized Low MW Hyaluronic acid (8-15 KDalton)

The sedimented gel in the centrifuge is washed by vortexing with 5 mL of EtOH/H₂O 4:1. It is centrifuged as before, obtaining the supernatant (surn2). Washing is repeated several times with 4 mL of EtOH, obtaining the corresponding supernatants, which are analysed by UV to determine the amount of PO_{DG} present that has not bound (% in Col M). In cases where the supernatants from the washes appeared slightly opalescent, indicating a possible colloidal dispersion unsuitable for UV analysis, 30 mg of finely pulverised NaCl were added and re-centrifuged again at 4000 g for 10 min, then UV-analysed. After the last washing, the gel is hydrated with 5 mL H₂O overnight. It is then frozen at −20° C. and freeze-dried. The results of the UV analysis of the supernatants are shown in Table 2.

TABLE 2
Results of UV analysis indicating the mg of PO_{DG} contained in the different
supernatants (ie PO_{DG} that did not react and crosslinked) and by difference with
the loaded PO_{DG} the amount of PO_{DG} crosslinked

| | | | mg PO_{DG} | | |
|---|---|---|---|---|---|
| | | Total mL | totali | | |
| PR006 | surn1 | 16.00 | 24.4 | | |
| | surn2 | 5.00 | 0.63 | | |
| | surn3 | 3.60 | 0.10 | | |
| | | total | 25.2 | % PO_{DG} recovered | % PO_{DG} |
| | | PO_{DG} mg | | (unreacted) = 100 | crosslinked = 0 |

| | | Total mL | mg totali | | |
|---|---|---|---|---|---|
| PR007 | surn1 | 7.25 | 14.6 | | |
| | surn2 | 5.40 | 1.54 | | |
| | surn3 | 4.25 | 1.54 | | |
| | | total | 17.6 | % PO_{DG} recovered | % PO_{DG} |
| | | PO_{DG} mg | | (unreacted) = 71 | crosslinked = 29 |
| | | Total mL | mg totali | | |
| PR008 | surn1 | 7.50 | 15.0 | | |
| | surn2 | 5.10 | 0.76 | | |
| | surn3 | 4.00 | 0.46 | | |
| | surn4 | 4.60 | 0.10 | | |
| | surn5 | 4.20 | 0.06 | | |
| | surn6 | 4.20 | 0.04 | | |
| | surn7 | 4.20 | 0.005 | | |
| | | total | 16.4 | % PO_{DG} recovered | % PO_{DG} |
| | | PO_{DG} mg | | (unreacted) = 66 | crosslinked = 44 |
| | | Total mL | mg totali | | |
| PR009 | surn1 | 14.50 | 15.3 | | |
| | surn2 | 5.25 | 0.28 | | |
| | surn3 | 4.25 | 0.05 | | |
| | surn4 | 4.00 | 0.02 | | |
| | surn5 | 4.25 | 0.01 | | |
| | | total | 15.66 | % PO_{DG} recovered | % PO_{DG} |
| | | PO_{DG} mg | | (unreacted) = 63 | crosslinked = 47 |

| | | Total mL | mg totali | | |
|---|---|---|---|---|---|
| PR010 | surn1 | 15.00 | 23.1 | | |
| | surn2 | 5.00 | 0.44 | | |
| | surn3 | 4.50 | 0.08 | | |
| | surn4 | 4.50 | 0.04 | | |
| | | total | 23.70 | % PO_{DG} recovered | % PO_{DG} |
| | | PO_{DG} mg | | (unreacted) = 95 | crosslinked = 5 |

| | | Total mL | mg totali | | |
|---|---|---|---|---|---|
| PR012 | surn1 | 6 | 11.30 | | |
| | surn2 | 5.5 | 5.16 | | |
| | surn3 | 4.2 | 2.66 | | |
| | surn4 | 4.3 | 3.96 | | |
| | surn5 | 4 | 1.44 | | |
| | | total | 24.53 | % PO_{DG} recovered | % PO_{DG} |
| | | PO_{DG} mg | | (unreacted) = 49 | crosslinked = 51 |
| | | Total mL | mg totali | | |
| PR013 | surn1 | 6.2 | 27.45 | | |
| | surn2 | 5.3 | 6.71 | | |
| | surn3 | 4.3 | 3.70 | | |
| | surn4 | 4.25 | 3.91 | | |
| | surn5 | 4.25 | 1.82 | | |
| | | total | 43.59 | % PO_{DG} recovered | % PO_{DG} |
| | | PO_{DG} mg | | (unreacted) = 56 | crosslinked = 44 |

| | | mL totali | mg totali | | |
|---|---|---|---|---|---|
| PR015 | surn1 | 5.0 | 31.3 | | |
| | surn2 | 5.6 | 11.4 | | |
| | surn3 | 4.5 | 7.0 | | |
| | surn4 | 4.5 | 4.9 | | |
| | surn5 | 4.5 | 3.5 | | |
| | surn6 | 4.5 | 2.3 | | |
| | surn7 | 4.2 | 1.9 | | |
| | surn8 | 4.1 | 4.0 | | |
| | surn9 | 4.0 | 2.1 | | |
| | surn10 | 4.1 | 0.8 | | |
| | | totale | 69.1 | % PO_{DG} recovered | % PO_{DG} |
| | | PO_{DG} mg | | (unreacted) = 55 | crosslinked = 45 |
| | | mL totali | mg totali | | |
| PR019 | surn1 | 7.25 | 92.0 | | |
| | surn2 | 5.25 | 5.8 | | |
| | surn3 | 4.30 | 0.7 | | |
| | surn4 | 4.00 | 0.5 | | |
| | surn5 | 4.20 | 0.2 | | |
| | surn6 | 4.20 | 0.1 | | |
| | surn7 | 4.00 | 0.1 | | |
| | surn8 | 4.00 | 0.0 | | |
| | surn9 | 4.50 | 0.1 | | |
| | surn10 | 4.20 | 0.0 | | |

| | | | |
|---|---|---|---|
| totale PO_{DG} mg | 99.6 | % PO_{DG} recovered | % PO_{DG} |
| | | (unreacted) = 80 | crosslinked = 20 |

UV Analysis Utilized for the Quantitation of PO_{DG} in the reaction of reticulation of HA with PO_{DG}

Readings were taken at λ=322 nm, for surn1 and 2 the reading was taken against a 4:1 mixture of EtOH/H2O, for sum3 and later against EtOH, using the working curves below reported (FIGS. A-C)

General Procedure for the Crosslinking Reaction Using Chloroacetylated Compounds: Hyaluronic Acid Cross-Linked with Polydatin Hexa-Chloroacetyl Derivative (Perchloroacetylated Polydatin)

Hyaluronic acid-sodium salt (HA) of intermediate molecular weight (MMW, 500-750 kDa) or low molecular weight (LMW, 8-15 kDa) were used. The cross-linking reactions were carried out using hyaluronic acid concentrations of 25 and 50 mg/mL for both. The tests performed were below reported as detailed in the “General Procedure” and in the table 1. The supernatants (surns) after washes and centrifugations were analysed to evaluate the total mg of perchloroacetylated Polydatin (POca) total present indicating the amount of cross-linker that did not bind and by difference the crosslinked POca on HA. The results are shown in Table 2.

### General Procedure

100 mg hyaluronic acid sodium salt (HA) is added to 2 or 4 mL of H₂O and the mixture is vortexed and left 15 min at 70° C. A solution of 0, 21, 42 mg of perchloroacetylated polydatin (POca) dissolved in 2 or 4 mL of DMSO is then added. The POca:HANa (repeat unit) mole ratios are given in Column F (they are 1:5 or 1:10). The mixture is heated under stirring at 70° C. for 15 hours. Then cooled to room temperature and 5 or 10 mL of EtOH or 10 mL EtOH+2 CH₃CN (Col I) are added to precipitate the polymer, vortexed for 2 minutes and centrifuge at 4000 g for 10 min. The supernatant (surn1) is analysed in the UV spectrophotometer to determine the mg of POca present. The gel is washed by vortexing with 5 mL of EtOH/H₂O 4:1 or directly with 4 mL CN₃CN. It is centrifuged as before, obtaining the supernatant (surn2). The wash is repeated several times with 4 mL of EtOH to obtain the corresponding supernatants, which are analysed by UV to determine the amount of POca present that has not bound (% in P column, Table 1′). In some cases, washing with 4 mL of CH₃CN in which POca is more soluble. In cases where the supernatants of the washes appeared slightly opalescent, indicating a possible colloidal dispersion, unsuitable for UV analysis, 30 mg of finely pulverised NaCl was added and re-centrifuged again at 4000 g for 10 min, and then proceeded with UV analysis. After the last wash, the gel is hydrated with 5 or 10 or 20 mL of H₂O overnight. It is then frozen in the freezer at −20° C. and freeze-dried.

The results of the UV analysis quantitation of POca of the supernatants are shown in Table 2′.

TABLE 1′
reaction conditions

| | | | | A | | C | D | E | F |
|---|---|---|---|---|---|---|---|---|---|
| | | | mg | mL | B | mg | mL | % cross- | Ratio^{b} |
| Entry | Sample | HA | HA | H₂O | mg/mL | POca | DMSO | linker^{a} | POca/HA |
| 0 | PR030A | MMW | 100 | 2 | 50 | 0 | 2 | — | — |
| 0 | PR030B | — | — | 2 | — | 42 | 2 | — | — |
| 1 | PR028A | MMW | 100 | 2 | 50 | 42 | 2 | 20 | 1:5 |
| 2 | PR028B | MMW | 100 | 4 | 25 | 42 | 4 | 20 | 1:5 |
| 3 | PR028C | LMW | 100 | 2 | 50 | 42 | 2 | 20 | 1:5 |
| 4 | PR028D | LMW | 100 | 4 | 25 | 42 | 4 | 20 | 1:5 |
| 5 | PR032A | MMW | 100 | 2 | 50 | 21 | 2 | 10 | 1:10 |
| 6 | PR032B | MMW | 100 | 2 | 50 | 42 | 2 | 20 | 1:5 |
| 7 | PR032C | LMW | 100 | 2 | 50 | 21 | 2 | 10 | 1:10 |
| 8 | PR032D | LMW | 100 | 2 | 50 | 42 | 2 | 20 | 1:5 |

| | | | | L | | O | |
|---|---|---|---|---|---|---|---|
| | | | | Washing 2 | M | Weight of the | |
| | G | H | I | mL | Washing 3 | freeze-dried | P |
| | t | Time | washing 1 | EtOH/H2O | and next | sample | % Bounded |
| Entry | ° C. | (hours) | mL EtOH | 4:1 | mL EtOH | mg | POca |
| 0 | 70 | 15 | 5 | 5 | 4 | 71.4 | — |
| 0 | 70 | 15 | 10 | — | — | — | — |
| 1 | 70 | 15 | 5 | 5 | 4 | 159 | 80 |
| 2 | 70 | 15 | 10 | 5 | 4 | 141 | 77 |
| 3 | 70 | 15 | 15 | 5 | 4 | | |
| 4 | 70 | 15 | 15 | 5 | 4 | | |
| 5 | 70 | 15 | 10 | 5 | 4** | 95 | 84 |
| 6 | 70 | 15 | 10 | 5 | 4** | 135 | 77 |
| 7 | 70 | 15 | 12* | — | 4* | | 80 |
| 8 | 70 | 15 | 12* | — | 4* | | 81 |

^{a}% cross-linker = mol POca/mol HA (repeated units) × 100
^{b}ratio = mol POca/mol HA (repeated units)
*the precipitation was done with 12 mL EtOH:CH₃CN (10:2, v/v) all the surnatants were obtained using 4 mL of CH₃CN Instead of EtOH.
**surnatants 7, 8, 9 were obtained using 4 mL of CH₃CN instead of EtOH.

TABLE 2′
UV analysis Quantitation of POca present in the supernatants
of the washing and the quantity of POca crosslinked to HA

| | | mL | mg | | |
|---|---|---|---|---|---|
| | | total | total | | |
| PR028A | surn1 | 6.3 | 4.7 | | |
| | surn2 | 5.5 | 1.3 | | |
| | surn3 | 4.5 | 0.7 | | |
| | surn4 | 4.4 | 0.8 | | |
| | surn5 | 4.2 | 0.4 | | |
| | surn6 | 4.2 | 0.2 | | |
| | surn7 | 4.0 | 0.2 | | |
| | surn8 | 4.2 | 0.1 | | |
| | surn9 | 4.5 | 0.1 | | |
| | surn10 | 4.5 | 0.1 | POca loaded in | 42.0 |
| | | | | the reaction mg | |
| | | Total | 8.5 | POca cross-linked | 33.5 |
| | | POca mg | | to HA mg | |
| PR028B | surn1 | 6.8 | 2.6 | | |
| | surn2 | 4.0 | 1.0 | | |
| | surn3 | 6.5 | 1.2 | | |
| | surn4 | 6.0 | 1.2 | | |
| | surn5 | 5.5 | 1.0 | | |
| | surn6 | 4.7 | 0.7 | | |
| | surn7 | 4.9 | 0.7 | | |
| | surn8 | 4.2 | 0.5 | | |
| | surn9 | 4.5 | 0.5 | | |
| | surn10 | 4.5 | 0.4 | POca loaded in | 42 |
| | | | | the reaction mg | |
| | | totale | 9.8 | POca cross-linked | 32.2 |
| | | POca mg | | to HA | |
| PR032A | surn1 | 11.0 | 1.57 | | |
| | surn2 | 5.0 | 0.4 | | |
| | surn3 | 5.0 | 0.53 | | |
| | surn4 | 4.2 | 0.2 | | |
| | surn5 | 4.2 | 0.23 | | |
| | surn6 | 4.5 | 0.1 | | |
| | surn7 | 4.5 | 0.19 | | |
| | surn8 | 4.2 | 0.06 | | |
| | surn9 | 4.3 | 0.02 | | |
| | surn10 | 0 | 0 | POca loaded in | 21 |
| | | | | the reaction mg | |
| | | totale | 3.3 | POca cross-linked | 17.7 |
| | | POca mg | | to HA | |
| PR032B | surn1 | 11.2 | 4.15 | | |
| | surn2 | 5 | 1.43 | | |
| | surn3 | 4.6 | 1.42 | | |
| | surn4 | 4.2 | 1.06 | | |
| | surn5 | 4.2 | 0.40 | | |
| | surn6 | 4.2 | 0.20 | | |
| | surn7 | 4.1 | 0.81 | | |
| | surn8 | 4.1 | 0.18 | | |
| | surn9 | 4.3 | 0.06 | | |
| | surn10 | 0.00 | 0.0 | POca loaded in | 42 |
| | | | | the reaction mg | |
| | | total | 9.8 | POca cross-linked | 32.2 |
| | | POca mg | | to HA | |
| PR032C | surn1 | 14.0 | 3.30 | | |
| | surn2 | 4.8 | 0.33 | | |
| | surn3 | 4.0 | 0.30 | | |
| | surn4 | 4.3 | 0.12 | | |
| | surn5 | 4.0 | 0.03 | | |
| | surn6 | 4.2 | 0.06 | | |
| | surn7 | 4.3 | 0.00 | | |
| | surn8 | 4.0 | 0.01 | | |
| | surn9 | 4.3 | 0.01 | | |
| | surn10 | 4.5 | 0.01 | POca loaded in | 21 |
| | | | | the reaction mg | |
| | | totale | 4.2 | POca cross-linked | 16.8 |
| | | POca mg | | to HA | |
| PR032D | surn1 | 15.0 | 5.60 | | |
| | surn2 | 4.3 | 0.48 | | |
| | surn3 | 4.5 | 0.49 | | |
| | surn4 | 4.5 | 0.45 | | |
| | surn5 | 4.3 | 0.27 | | |
| | surn6 | 4.5 | 0.20 | | |
| | surn7 | 4.1 | 0.28 | | |
| | surn8 | 4.5 | 0.05 | | |
| | surn9 | 4.3 | 0.04 | | |
| | surn10 | 4.1 | 0.03 | POca reacted mg | 42 |
| | | total | 7.9 | POca legbound mg | 34.1 |
| | | POca mg | | | |

UV Analysis Utilized for the Quantitation of POca in the Reaction of Reticulation of HA with POca

Readings were taken at λ=313 nm, for surn1 and 2 the reading was taken against a 4:1 EtOHIH2O mixture, for sumn3 and following against EtOH, using the working curves shown below. In the cases shown in the table it was done against EtOH:CH3CN 10:2 or against CH3CN (see FIGS. A′-E′)

Analyses in HPLC_SEC of the Obtained Fillers: PO_{DG} Crosslinked with Hyaluronic Acid

Hyaluronic acid, polydatin diglycidylated and the fillers batch numbers PR019, PR015 and PR008 were analysed by Size Exclusion Chromatography (SEC) using an HPLC Column BioSep (5 um SEC-s3000 290A) and as mobile phase H₂O; flow rate 1 mL/min. Medium molecular weight HA is injected at a concentration of 0.5 mg/mL (1 uL, 5 uL, 10 uL, 25 uL) and the spectrum is acquired at =322 nm (at this wavelength PO_{DG} absorption is maximum and little HA), and at 220 nm and 215 nm where HA absorption is maximum. 25 uL of these solutions were injected; we then proceed by injecting some prepared samples, suitably solubilised at the concentration of 0.5 mg/mL.

TABLE 1″

| | | HA Area | Ha Area | Ha Area | |
|---|---|---|---|---|---|
| | | (tr = | (tr = | (tr = | Area |
| | Injected | 4 min), | 4 min), | 4 min), | ratio |
| | volume | λ = 322 | λ = 220 | λ = 215 | 220/322 |
| HAm | 1 uL | 129 | 557 | 653 | 4.3 |
| 0.5 mg/mL | | | | | |
| | 5 uL | 258 | 1083 | 1268 | 4.1 |
| | 10 uL | 366 | 1448 | 1780 | 3.95 |
| | 25 uL | 637 | 2517 | 3155 | 3.74 |
| PR0006A | 25 uL | 609 | 2330 | 2873 | 3.83 |
| 0.5 mg/mL | | | | | |
| PR0007A | 25 uL | 678 | 2811 | 3504 | 4.15 |
| 0.5 mg/mL | | | | | |
| PR015 | 25 uL | 3417 | 7261 | 8354 | 2.12 |
| 0.5 mg/mL | | | | | |
| PR008 | 25 uL | 1577 | 4477 | 5142 | 2.83 |
| 0.5 mg/mL | | | | | |
| HAl | 25 uL | 637 | 2517 | 3155 | 3.74 |
| 0.5 mg/mL | | | | | |
| PR019 | 25 uL | 5098 | 9261 | 10236 | 2.12 |
| 0.5 mg/mL | | | | | |
| PR019 | 25 uL | 4245 | 4477 | 5142 | 2.83 |
| 0.5 mg/mL | | | | | |
| (new) | | | | | |

The results, reported in Table 1″, confirmed that the crosslinked derivatives of HA with PO_{DG} batch numbers PR015, PR008 and PR019 contains a sensible amount of polydatin and further support the found percentage of polydatin present in the filler reported in table 2.

NMR Evaluation of the Degree of Reticulation of the Fillers Obtained by Reaction of Hyaluronic Acid (Different Molecular Weights) with Polydatin Hexachloroacetyl Derivative (POca)

The degree of reticulation of the fillers obtained by the reaction of hyaluronic acid (different molecular weights) with polydatin hexachloroacetyl derivative (POca) was also studied using NMR spectroscopy of the fillers after hydrolysis carried out in deuterated sodium hydroxide (NaOD): the results are expressed as a ratio between the CH₃-related signals of the N-acetylglucosamine of hyaluronic acid (HA) (d 1.80±0.5 ppm) and the protons in trans (J_{H,H}=16.0) on the double bond of polydatin (PO) at 6.93±0.5 ppm e 6.67±0.5 ppm (as shown in ¹H-NMR spectra 1 for the filler PR032D). The data reported in table 2′) further support the outcome of the percentage of bounded polydatin on hyaluronic acid reported in table 1′.

The NMR data confirm the assumption that:
the obtained fillers are crosslinked with a high degree of acetylated polydatin since a strong basic hydrolysis releases from the fillers polydatin (PO) and hyaluronic acid (HA) in a relative ratio in agreement with those reported in table 1′ (% of bound POca).
that the higher degree of reticulation of these fillers were obtained using a relative molar ratio between POca and HA (for HA the moles taken into account refer to the repeated disaccharide units of glucuronic acid sodium salt and N-acetylglucosamine) of 1:5 respect to 1:10, both on MMW and LMW hyaluronic acids.

### Materials and Methods

3 mg of the filler were solubilized in 0.7 mL of 0.5 M NaOD (in some experiments, samples were also solubilised in 0.25 M NaOD with no substantial difference; working in more diluted conditions the solubilization time increased by a few minutes). NaOD was prepared at the concentration of 1M by solubilizing Na metal in D₂O in a suitable anhydrous vessel under an argon atmosphere and on ice (to avoid the possible interference of not deuterated water). The 1M NaOD solution was then diluted 1:1 v/v with D₂O to give the 0.5M final solution or to 1:4 v/v to give the 0.25M final solution. The ¹H-NMR (500 MHz) spectrum were acquired with a scan number greater than 16. The spectrum of the polymers was compared with that of medium-weight hyaluronic acid alone in NaOD 0.25M or NaOD 0.5M, with that of PR030A (reaction carried out without the addition of perchloroacetylated polydatin; see table 1′) in NaOD 0.5M, with polydatin alone or with perchloroacetylated polydatin alone in NaOD 0.5M (the latter two obviously coincide, as perchloroacetylated polydatin in NaOD 0.5M is hydrolysed into free polydatin).

The relationship between the CH₃-related signals of the N-acetylglucosamine of hyaluronic acid and the aromatic protons of polydatin was evaluated

The 1H-NMR spectra 1 is reported in FIG. F from the top to the bottom HA in NaOD solution, polydatin in NaOD solution and Filler PR032D

### Results

TABLE 2″

| | | | | | NMR |
|---|---|---|---|---|---|
| | | % cross- | Ratio^{b} | % | analyses |
| Sample | HA | linker^{a} | POca/HA | BoundPOca | HA/PO^{c} |
| PR028A | MMW | 20 | 1:5 | 80 | 1:1 |
| PR028B | MMW | 20 | 1:5 | 77 | 1:0.8 |
| PR032A | MMW | 10 | 1:10 | 84 | 1:0.17 |
| PR032C | LMW | 10 | 1:10 | 80 | 1:0.33 |
| PR032D | LMW | 20 | 1:5 | 81 | 1:0.8 |

^{a}% cross-linker = mol POca/mol HA (repeated units) × 100
^{b}ratio = mol POca/mol HA (repeated units)
^{c}the relationship between the CH₃-related signals of the N-acetylglucosamine of hyaluronic acid (HA) and the aromatic protons of polydatin (PO) were evaluated by mean of ¹H-NMR spectroscopy after hydrolysis of the filler samples in NaOD solution.

1. Hyaluronic acids cross-linked with activated derivatives of plant polyphenols selected from polydatin, gallic acid, chlorogenic acid, ellagic acid and phloridzin.

2. Cross-linked hyaluronic acids according to claim 1, wherein the activated derivatives of the plant polyphenols are glycidyl ethers (oxiran-2-yl-methyl ethers) or 2-chloroacetyl esters.

3. Cross-linked hyaluronic acids according to claim 2, wherein the activated derivative is selected from diglycidated polydatin, hexa-chloroacetyl polydatin, a mixture of mono-, di-, tri- and tetra 2-chloroacetyl polydatin esters, 3,4,5-tris(2-chloroacetyl) esters of gallic acid, oxiran-2-ylmethyl 3,4,5-tris(oxiran-2-ylmethoxy) benzoate, hepta-2-chloroacetyl derivative of phlorizin, penta-chloroacetyl derivative of chlorogenic acid, oxiran-2-ylmethyl 3,4,5-tris(oxiran-2-ylmethoxy) benzoate and 3,4,5-tris(2-chloroacetoxy)benzoic acid,

4. Cross-linked hyaluronic acids according to claim 3, wherein the activated derivatives are glycidyl ethers or 2-chloroacetyl esters of polydatin or gallic acid.

5. Cross-linked hyaluronic acids according to claim 3, wherein the relative molar ratio of mono-, di-, tri- and tetra-2-chloroacetyl polydatin esters is 17±3.4%/44.3±8.8%/17.7±3.6%/1.9±0.4%.

6. Cross-linked hyaluronic acids according to claim 1, obtained from hyaluronic acid having average molecular weight Mn of 80-110 kDa.

7. Cross-linked hyaluronic acids according to claim 1, obtained from hyaluronic acid having average molecular weight Mn of 250-450 kDa.

8. Crosslinked hyaluronic acids according to claim 1, obtained from hyaluronic acid having average molecular weight Mn of 1.5-3.0 MDa.

9. Cross-linked hyaluronic acids according to claim 1, having a degree of cross-linking such as to give a ratio of viscous modulus (G″) to elastic modulus (G′) of less than 1.0.

10. A process for preparing cross-linked hyaluronic acids of claim 1, said process comprising
reacting cross-linked hyaluronic acid in aqueous solution with activated derivatives of plant polyphenols in dimethyl sulfoxide solution at a temperature ranging from 30 to 80° C.

11. Process according to claim 10, wherein the molar ratio of activated polyphenol derivative to hyaluronic acid ranges from 1:1 to 1:10.

12. Intradermal or intra-articular injectable compositions in the form of sterile gels comprising the cross-linked hyaluronic acids of claim 1.

13. Injectable compositions according to claim 12, comprising a mixture of cross-linked hyaluronic acids of different molecular weights.

14. Injectable compositions according to claim 13, comprising the cross-linked hyaluronic acids obtained from hyaluronic acid having average molecular weight Mn of 80-110 kDa, 250-450 kDa or 1.5-3.0 MDa.

15. Injectable compositions according to claim 12, comprising
1 mg/ml to 50 mg/ml of crosslinked hyaluronic acids, optionally in the presence of an anaesthetic, in a concentration of 0.1 to 0.4% weight/volume.

16. The compound (2R,3S,4S,5R,6S)-2-(hydroxymethyl)-6-(3-(oxiran-2-ylmethoxy)-5-((E)-4-(oxiran-2-ylmethoxy) styryl) phenoxy) tetrahydro-2H-pyran-3,4,5-triol of formula

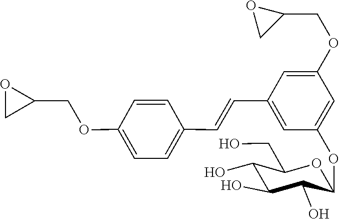

17. Polydatin hexa-2 chloroacetyl of formula

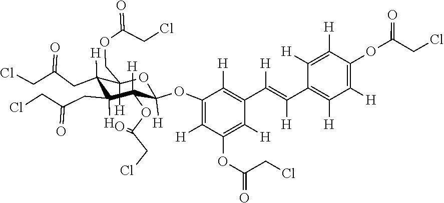

18. Hepta-2-chloroacetyl phlorizin of formula

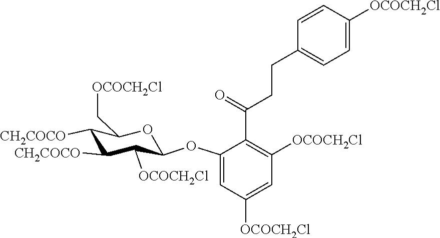

19. Penta-2-chloroacetyl chlorogenic acid of formula

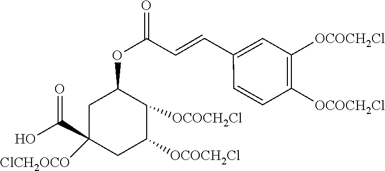

20. The process according to claim 10, wherein the temperature ranges from 50 to 80° C.

21. The injectable compositions according to claim 15, wherein the anaesthetic is lidocaine.
